(19) United States
(12) Patent Application Publication
(10) Pub. No.: US 2014/0228648 A1
(43) Pub. Date: Aug. 14, 2014

(54) PET SCALE KIOSK WITH INTERACTIVE CLIENT INFORMATION SOFTWARE

(71) Applicant: BAG ENTERPRISES, INC, (US)

(72) Inventors: BARRY A. GOLDBERG, COCONUT GROVE, FL (US); KIMBERLY KULHANJIAN, PLANTATION, FL (US)

(73) Assignee: BAG ENTERPRISES, INC., MIAMI, FL (US)

(21) Appl. No.: 13/839,405

(22) Filed: Mar. 15, 2013

Related U.S. Application Data
(60) Provisional application No. 61/764,356, filed on Feb. 13, 2013.

(51) Int. Cl. A61B 5/107; A61B 3/00; A61B 5/0205; A61B 5/12; A61B 5/00; A61B 5/01

(52) U.S. Cl. CPC A61B 5/107; A61B 5/0002; A61B 5/01; A61B 5/0205; A61B 5/441; A61B 5/4547; A61B 5/12; A61B 3/00
USPC 600/301; 600/587

(57) ABSTRACT

A method of pet health monitoring using an interactive computerized kiosk communicably connected to a scale includes a petcare client placing their pet on the scale, wherein the interactive computerized kiosk obtains the pet's weight. The petcare client interacts with the interactive computerized kiosk, wherein the petcare client provides pet data regarding their pet responsive to questions posed by the interactive computerized kiosk for gathering the pet data. The interactive computerized kiosk automatically generates at least one health related condition assessment based on the pet's weight and the pet data, and at least one pet-related coupon for pet products or pet services is provided to the petcare client.

### CROSS REFERENCE TO RELATED APPLICATIONS

This application claims the benefit of Provisional Application Ser. No. 61/764,356, entitled “PET SCALE KIOSK WITH INTERACTIVE CLIENT INFORMATION SOFTWARE”, filed Feb. 13, 2013, which is herein incorporated by reference in its entirety.

### FIELD

Disclosed embodiments relate to kiosks for retailing petcare products.

### BACKGROUND

In the field of retailing petcare products, there is a need for more effective marketing.

### SUMMARY

This Summary is provided to introduce a brief selection of disclosed concepts in a simplified form that are further described below in the Detailed Description including the drawings provided. This Summary is not intended to limit the claimed subject matter's scope.

Disclosed embodiments include methods of pet health monitoring using an interactive computerized kiosk communicably coupled to a scale that includes a petcare client placing their pet on the scale. The interactive computerized kiosk obtains the pet's weight. The petcare client interacts with the interactive computerized kiosk, wherein the petcare client provides pet data regarding their pet responsive to questions posed by the interactive computerized kiosk for gathering the pet data. The interactive computerized kiosk generates at least one health related condition assessment based on the pet's weight and the pet data, and at least one pet-related coupon for pet products or pet services is provided to the petcare client.

Disclosed embodiments also include interactive computerized kiosks which can include a scale. A computing system typically having a touchscreen monitor can implement disclosed client interactive software which obtains petcare client' information (e.g., email and physical address) and pet information for the petcare client's pet. Based on the pet information received the software evaluates the pet's weight utilizing a pet weight chart that allows clients in pet stores (and other stores) to evaluate if their pet is at a healthy weight, and can also educate clients on ways to prevent pet obesity and correct a problem if determined to be needed.

Designed as a free standing and interactive computerized kiosk, disclosed embodiments represent a new approach to keeping pets healthy. Deployed in retail chains and stores creates a convenient way to monitor a pet's health, and allows the pet owner to consult with a real veterinarian (e.g., via video communication) regarding any possible health concerns. Monitoring a pet's health regularly will allow the petcare owner a greater chance at recognizing, treating any avoiding harmful health issues that may be affecting the pet.

### BRIEF DESCRIPTION OF THE DRAWINGS

FIG. 1 is a flow chart that shows steps in an example method of pet health monitoring using an interactive computerized kiosk communicably connected to a scale that provides a weight of a petcare client's pet, according to an example embodiment.

FIG. 2 shows a perspective view depiction of an example standalone petscale computerized kiosk with a computing system implementing disclosed interactive client information software and skip logic software, according to an example embodiment.

FIG. 3A is a depiction of an example interactive computerized kiosk that unlike the interactive computerized kiosk shown in FIG. 2, does not include a scale; according to an example embodiment; FIG. 3B is a perspective view of the interactive computerized kiosk shown in FIG. 3A, with its top removed to show inner features; FIG. 3C shows a depiction of the inside of the interactive computerized kiosk shown in FIG. 3A, and FIG. 3D shows an example layout for the interactive computerized kiosk shown in FIG. 3A.

### DETAILED DESCRIPTION

Disclosed embodiments in this Disclosure are described with reference to the attached figures, wherein like reference numerals are used throughout the figures to designate similar or equivalent elements. The figures are not drawn to scale and they are provided merely to illustrate the disclosed embodiments. Several aspects are described below with reference to example applications for illustration. It should be understood that numerous specific details, relationships, and methods are set forth to provide a full understanding of the disclosed embodiments. One having ordinary skill in the relevant art, however, will readily recognize that the subject matter disclosed herein can be practiced without one or more of the specific details or with other methods. In other instances, well-known structures or operations are not shown in detail to avoid obscuring structures or operations that are not well-known. This Disclosure is not limited by the illustrated ordering of acts or events, as some acts may occur in different orders and/or concurrently with other acts or events. Furthermore, not all illustrated acts or events are required to implement a methodology in accordance with this Disclosure.

Disclosed interactive computerized kiosks are interactive, self-service veterinary health-screening kiosks that can service pets including dogs, cats and small “pocket pets”, and offers pet owners convenient access to non-invasive preventative and wellness veterinary care, consultations, and can provide a referral system for veterinary doctors, specialists and other veterinary related services. Petcare clients can receive their pet's customized health screening results for their pet, an overall health assessment and access to a database of local veterinarians. Disclosed software can email the pet's medical results to a specified veterinarian for further consultation or treatment, as well as set up an account to track their pet's medical results online, which in one embodiment all can be accessed via a user-friendly, touch screen interface. Disclosed kiosks can evaluate a variety of vital health parameters utilizing cutting edge technology and software that communicates directly and in near real-time to an attending Doctor of Veterinary Medicine (DVM) that can host the physical exam via a private video conferencing system:

Example diagnostic services include obtaining pet health parameters including heart rate (by a heart rate monitor), blood pressure (by a BP monitor), respiration (visual by a technician), temperature (by a thermometer), weight and body mass index (by a scale), ear examination (visual and by an otoscope), eye examination (visual and by an otoscope), visual abnormalities on the skin, teeth and body structure. A health risk assessment can be rendered after evaluation by a professional (e.g., a remotely located DVM that teleconferences with the petcare client) based on the pet health parameters, and history and physical information regarding the pet gained from the petcare client.

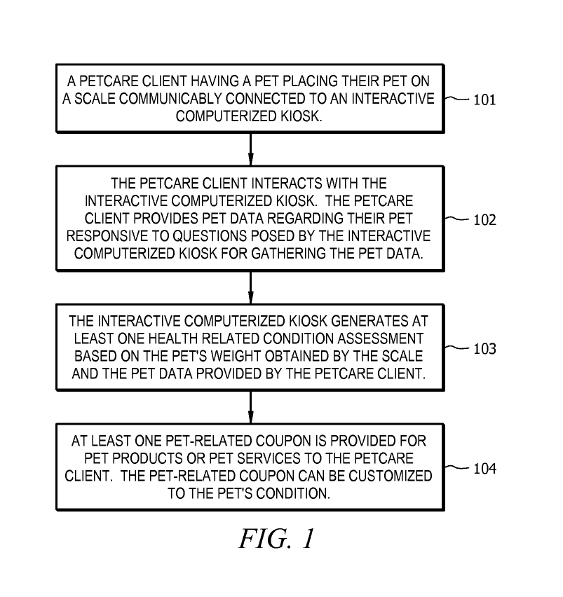

FIG. 1 is a flow chart that shows steps in an example method 100 of pet health monitoring using an interactive computerized kiosk communicably connected to a scale that provides a weight of a petcare client's pet (pet's weight), according to an example embodiment. Disclosed interactive computerized kiosks can be located anywhere pets are allowed, typically a retail store pet store or other store (department store), or other locations such as when embodied as a mobile clinic on wheels. The interactive computerized kiosk can provide the scale for providing the pet's weight, or the scale can be a remotely locate scale somewhere in the building with the interactive computerized kiosk that is communicably connected to the interactive computerized kiosk.

The pet's weight is determined by weighing the pet on the scale. In the embodiment the interactive computerized kiosk includes a scale, the petcare client and their pet can approach the interactive computerized kiosk; and the petcare client can be told on a display screen of the kiosk or by a speaker to place their pet on the scale. Step 101 comprises a petcare client having a pet placing their pet on the scale, where the interactive computerized kiosk obtains the pet's weight. The scale can provide a digital output in one embodiment.

Step 102 comprises the petcare client interacting with the interactive computerized kiosk. The petcare client provides pet data (e.g., via a touch screen of the interactive computerized kiosk or by voice recognition) regarding their pet responsive to questions posed by the interactive computerized kiosk for gathering the pet data. Memory at the interactive computerized kiosk can store the pet data received and can build a database with pet data for each specific pet including a date stamp, which can be sent over a network to all interactive computerized kiosks in the network, and also be sent for remote storage (e.g., cloud storage).

Step 103 comprises the interactive computerized kiosk generates at least one health related condition assessment based on the pet's weight obtained by the scale and the pet data provided by the petcare client. The health risk assessment provided to the petcare client may be provided with supporting information for a variety of possible conditions or concerns, and can assess the pet as being underweight, ideal weight, overweight; such as by comparing the pet's attributes provided by the pet data (e.g., an adult female akita) to the pet's weight. Disclosed embodiments recognize the pet's weight is generally an important indicator for medical problems of pets including for obesity, malnutrition and under-weight pathologies.

Step 104 comprises providing at least one pet-related coupon for pet products or pet services to the petcare client. The pet-related coupon can be customized to the pet's condition, and can be provided to petcare client by a variety of means, including by an email address of the petcare client provided to the interactive computerized kiosk in step 102, conventionally mailed to petcare's clients home or business, and/or printed locally in the location that the interactive computerized kiosk is situated (e.g., inside a store).

The interactive computerized kiosk can includes a plurality of pet examination devices. This allows the interactive computerized kiosk to provide guided assistance (on site or via virtual Internet/video conferencing communication) to the petcare client or an on-site petcare technician for utilizing the pet examination devices for measuring pet health parameters other than weight to begin evaluation of a plurality of non-weight health parameters. These health parameters can include, but are not limited to, heart rate, blood pressure, respiration, temperature, ear condition, eye condition, and condition of the skin, teeth, and body structure.

The kiosk can include communications equipment and a communications link enabling the petcare client to teleconference with a remote pet care professional, such as a board certified veterinarian (e.g., DVM). The teleconference can include videoconferencing with a veterinarian.

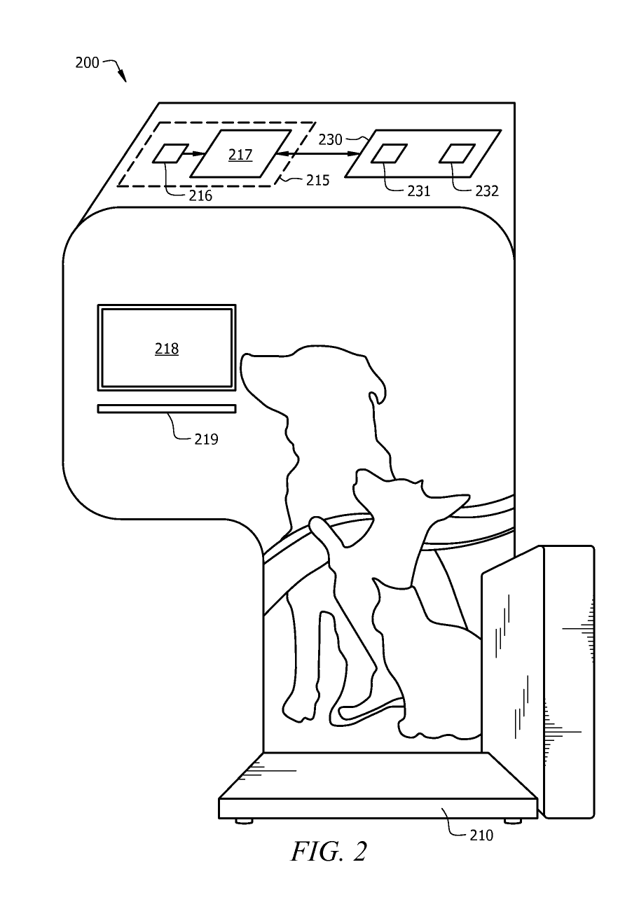

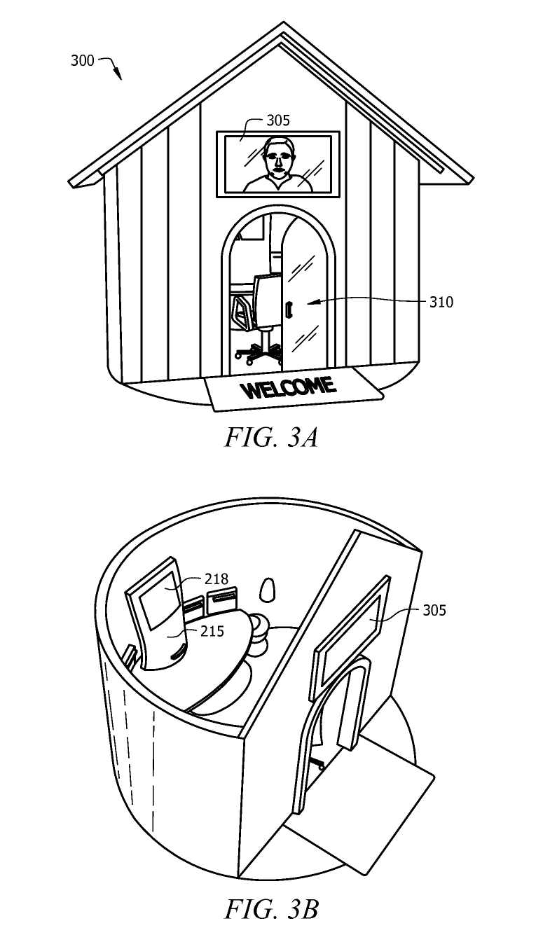

FIG. 2 shows a perspective view depiction of a standalone interactive computerized kiosk 200 including a computing system 215 implementing interactive client information software and skip logic software stored in memory 216, according to an example embodiment. The skip logic software is configured to generate skip logic questions and use petcare client' answers in response to these questions, including to narrow down to an accurate and appropriate weight chart for the pet. The interactive computerized kiosk includes a scale 210 adapted for weighing a pet (e.g., dog or cat) to obtain a pet's weight. Scale 210 can be a conventional scale, and provide a digital output. The scale can also be a scale that also measures body mass index (BMI) BMI is the most common obesity measure used for humans, which estimates body fatness based upon height and weight. A BMI can help determine if a pet's weight is normal.

Computing system 215 including a display 218, a processor (e.g., digital signal processor (DSP)) 217, and a memory 216 (e.g., static random access memory (SRAM) storing a database, and implementing disclosed interactive client software and skip logic software which the processor 217 implements. An optional keyboard 219 provides the user interface coupled to the computing system 215 which allows a petcare client to input answers to questions including questions regarding their pet posed on the display 218 by the interactive client software. A touchscreen monitor can also be used to provide the functions of both display 218 and keyboard 219. Disclosed skip logic software automatically determines a pet's weight status based on the pet's weight (i.e., charts—underweight, healthy weight or overweight), dynamically determines the next questions for the per care client, and can generate a final page which corresponds to a weight chart, based on answers previously provided by the petcare client.

Disclosed embodiments recognize weight gain or weight loss can be a significant identifier of a serious medical condition in pets. Even the gain/loss of just 2 to 3 pounds over the course of a few days can be the sign of a serious medical issue. Use of disclosed kiosks with interactive client information software can thus alert a client to a potentially serious medical issue while the client is shopping for petcare products in a pet store or other store. FIG. 2 also shows pet examination devices 230 for measuring pet health parameters other than the pet's weight comprising a first pet examination device 231, and a second pet examination device 232.

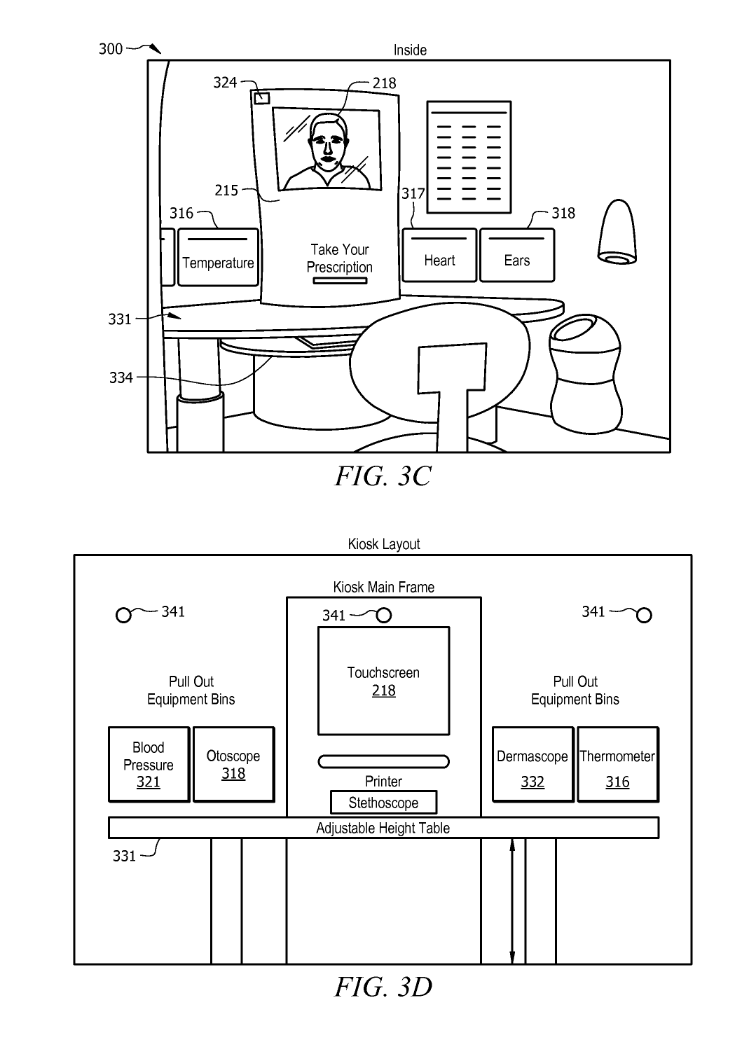

FIG. 3A is a depiction of an example interactive computerized kiosk 300 that unlike interactive computerized kiosk 200 shown in FIG. 2, does not include a scale. In this embodiment, the pet scale is remotely located from the interactive computerized kiosk 300, such as in another portion of the store, and the remotely located scale is communicably connected (e.g., an RF or Internet link) to the interactive computerized kiosk 300. As shown in FIG. 3A, the monitor 305 shown can be an LCD monitor that displays a welcome sign and the pet's weight. The automatic sliding door 310 shown can be provided. FIG. 3B is a perspective view of the interactive computerized kiosk 300 with its top removed to show inner features showing an example space-saving semicircular shape.

FIG. 3C shows a depiction of the inside of the interactive computerized kiosk 300. FIG. 3C shows example pet examination devices for measuring pet health parameters other than the pet's weight including a temperature monitoring device 316, a heart monitoring device 317 and an ear monitoring device (e.g., otoscope) 318. The scanned image depicted is that of a remotely located DVM displayed by the monitor 218, which is enabled by a communications link 324 for remote teleconferences. Interactive computerized kiosk 300 is shown in FIG. 3C having an adjustable table 331 and a retractable touchscreen table 334 for providing added controls and instructions.

FIG. 3D shows an example layout for interactive computerized kiosk 300. A video camera 341 is provided to facilitate the virtual interaction by teleconference (or videoconference), such as with a DVM. The example pet examination devices shown in FIG. 3D for measuring pet health parameters other than the pet's weight shown in FIG. 3D include a blood pressure monitor 321, dermascope 332, ear monitoring device (e.g., (otoscope) 318, and a temperature monitoring device 316.

### EXAMPLES

Disclosed embodiments are further illustrated by the following specific Examples, which should not be construed as limiting the scope or content of this Disclosure in any way.

Regarding implementing software, disclose software can utilize a Quick Tap Survey Program provided by TabbleDabble Inc., (or similar program) designed for market research, in-store customer feedback, lead capture, marketing and kiosk data collection. Disclosed software can create customized surveys to gather customer/pet information. The software can be accessed using a computer with touchscreen monitor to obtain client and pet information, evaluate pet's weight utilizing pet weight charts, make recommendations based off of the results, and generate coupons to market designated products or services related to a pet's condition.

Utilizing disclosed skip logic software system allows determining the pet's weight status using weight charts, underweight, healthy weight or overweight. The software can be programmed to dynamically determine the next questions, or the final page which can be the corresponding weight chart, based on answers previously provided by the petcare client. Accordingly, depending on the petcare client's answers, the software can provide the appropriate weight chart and recommendations. Information gathered by using touch screen options can include the pet's name, Type: Dog or Cat, Gender: Female or Male, Age: 0-6 months, Puppy/Kitten, 6 months to 8 years Adult, over 8 years Geriatric, and the petcare client's email and physical address. An automatic email can be sent to the client upon completion of survey questions with information and special offers. Additionally, the last page of the survey can have special in-store offers valid for instant redemption that may be printable, such as via a Wi-Fi printer attached to the kiosk.

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. In addition, while a particular feature may have been disclosed with respect to only one of several implementations, such feature may be combined with one or more other features of the other implementations as may be desired and advantageous for any given or particular application.

Thus, the breadth and scope of the subject matter provided in this Disclosure should not be limited by any of the above explicitly described embodiments. Rather, the scope of this Disclosure should be defined in accordance with the following claims and their equivalents.

1. A method of pet health monitoring, comprising:
providing a interactive computerized kiosk including a scale, wherein said scale provides a weight of a pet (pet's weight), said interactive computerized kiosk further comprising a computer, an interactive client interface and computer executable instructions such that when said instructions are executed:
presents multi-step instructions on said interactive client interface to prompt receipt of pet information other than said pet's weight including a type of animal for said pet;
generates at least one health related condition assessment based on information including said pet's weight compared to a weight standard, and
generates at least one pet-related coupon for pet products or pet services selected based on said health related condition assessment.

2. The method of claim 1, wherein said interactive computerized kiosk includes at least one pet examination device, and wherein said interactive computerized kiosk further provides guided assistance for utilizing said pet examination device for measuring pet health parameters other than said pet's weight.

3. The method of claim 2, wherein said pet health parameters other than said pet's weight which are generated include at least one of heart rate, blood pressure, respiration, temperature, ear examination, eye examination, visual abnormalities on skin, teeth, and body structure.

4. The method of claim 1, wherein said interactive computerized kiosk includes a communications link for remote teleconferencing with remotely located veterinarians.

5. The method of claim 1, wherein said scale is a remotely located scale positioned in a common building with said interactive computerized kiosk and is communicably connected for communicating with said interactive computerized kiosk.

6. The method of claim 1, wherein said pet-related coupon which is generated is customized to said condition assessment.

7. An interactive computerized kiosk, comprising:
a scale for providing a weight of a pet (pet's weight);
a computing system coupled to receive said pet's weight from said scale including a display, a processor, and a memory storing a database having interactive client software and skip logic software which said processor implements;
a user interface coupled to said computing system which allows a petcare client to provide pet data regarding their pet responsive to multi-step questions posed by said interactive client software for gathering the pet data including a type of animal for said pet;
wherein said skip logic software automatically generates at least one health related condition assessment based on information including said pet's weight compared to a weight standard, and
wherein said interactive computerized kiosk provides at least one pet-related coupon for pet products or pet services selected based on said health related condition assessment.

8. (canceled)

9. The interactive computerized kiosk of claim 7, wherein said interactive computerized kiosk further comprises at least one pet examination device for measuring pet health parameters other than said pet's weight.

10. The interactive computerized kiosk of claim 9, wherein said pet examination device include at least two of a heart rate monitor, a blood pressure monitor, a respiration monitor, a temperature monitor, and ear examination device, and eye examination device, a device for measuring visual abnormalities on skin, teeth, or body structure of said pet.

11. The interactive computerized kiosk of claim 7, wherein said interactive computerized kiosk includes a communications link for remote teleconferences.

12. (canceled)

13. The method of claim 1, wherein said pet data further includes at least one of a breed for said pet, a gender for said pet, and an age for said pet.

14. The method of claim 13, wherein said weight standard comprises an ideal weight for said pet obtained from a pet weight chart.

15. The method of claim 14, further comprising comparing said ideal weight to said pet's weight, wherein said health related condition assessment includes a determination of said pet being underweight, ideal weight, or overweight.

16. The method of claim 1, wherein said interactive computerized kiosk is within a retail store.

17. The interactive computerized kiosk of claim 7, wherein said pet data further includes at least one of a breed for said pet, a gender for said pet, and an age for said pet.

18. The interactive computerized kiosk of claim 7, wherein said weight standard comprises an ideal weight for said pet obtained from a pet weight chart.

19. The interactive computerized kiosk of claim 18, wherein said processor further implements comparing said ideal weight to said pet's weight, wherein said health related condition assessment includes a determination of said pet being underweight, ideal weight, or overweight.
